(19) United States
(12) Patent Application Publication
(10) Pub. No.: US 2013/0210035 A1
(43) Pub. Date: Aug. 15, 2013

(54) CHIP AND METHOD FOR DETECTING GLYCOSYLATED HEMOGLOBIN

(71) Applicants: Chih-Hsing WU, Tainan City (TW); Huang-Han CHEN, Tainan City (TW); Shu-Hui CHEN, Tainan City (TW); Mei-Ling Tsai, Tainan City (TW); Horng-Yih OU, Tainan City (TW)

(72) Inventors: Chih-Hsing WU, Tainan City (TW); Huang-Han CHEN, Tainan City (TW); Shu-Hui CHEN, Tainan City (TW); Mei-Ling Tsai, Tainan City (TW); Horng-Yih OU, Tainan City (TW)

(21) Appl. No.: 13/585,983

(22) Filed: Aug. 15, 2012

(30) Foreign Application Priority Data
Aug. 15, 2011 (TW) 100129110

(51) Int. Cl. G01N 33/66

(52) U.S. Cl. CPC G01N 33/66
USPC 435/7.92

(57) ABSTRACT

The present invention relates to a chip and a method for detecting HbA1c. The detection chip comprises: a substrate; and a biomolecular layer disposed on the substrate, wherein the biomolecular layer comprises a first anti-hemoglobin antibody. The biomolecular layer can bind both the glycosylated hemoglobin and the hemoglobin in the blood sample. The method for detecting HbA1c of the present invention comprises the use of a bio-detection layer that comprises anti-glycosylated hemoglobin antibody and a second hemoglobin antibody with different epitope to differentiate the glycosylated hemoglobin from the total hemoglobin. Thus, the relative amount of glycosylated hemoglobin to hemoglobin can be detected by using the detection chip and the detection method of the present invention.

### CROSS REFERENCE TO RELATED APPLICATION

This application claims the benefits of the Taiwan Patent Application Serial Number 100129110, filed on Aug. 15, 2011, the subject matter of which is incorporated herein by reference.

### BACKGROUND OF THE INVENTION

1. Field of the Invention

The present invention relates to a chip for detecting glycosylated hemoglobin and a method for preparing the same. More specifically, the present invention relates to a detecting chip applicable in showing average blood glucose concentration.

2. Description of Related Art

Based on the annual report conducted by the Taiwanese Department of Health, diabetes is now the number four leading cause of death in Taiwan, and is considered a disease with high mortality. Because diabetes can cause complications such as diabetic retinopathy, cardiovascular disease, renal failure, or neurological disorders and other diseases, which can further inflict damage to the brain and the circulatory system, therefore, effective blood glucose control has topped the list of priorities for the health care.

In an attempt to deal with the severity of diabetes, common practices including maintaining healthy eating habit or medical treatment has been demonstrated to allow gradually scaling the blood glucose level back to a normal range so as to lower the risk of diabetic complications. Therefore, the blood glucose measurement being recognized as one of important value for the early diagnosis or well control of diabetes. The traditional method of detecting blood glucose would require separate measurements taken from before or after meal intake, however, test results coming from such method are easily susceptible to fluctuation with respect to daily meals, precise blood glucose measurement results are therefore difficult to obtain.

Empirical studies have shown that concentration of glycosylated hemoglobin (HbA1c) would not undergo significant changes due to the current blood glucose concentration. After the reaction between glucose in blood and hemoglobin is complete, the two compounds will slowly merge to form glycosylated hemoglobin (HbA1c). An after-meal measurement would not instantly change the concentration of glycosylated hemoglobin since the combination of the two chemicals can remain in the body for a certain length of time. Due to this characteristic, HbA1c has been considered an important surrogate for evaluating blood glucose regulation, and is even considered a novel application in diagnosis of diabetes.

In another object of the present invention to enhance operability of using HbA1c as a diagnosis tool, a chip for detecting HbA1c and a method for detecting the same are provided herein, thereby providing patients with easier accessibility for diabetic care at home.

Even though sandwich immunoassay is currently claimed as having the highest degree of specificity, sensitivity, and stability repeatability, liquid chromatography or single antibody remains to be the most widely used clinical method for detecting glycosylated hemoglobin, for which one of the main contributing reasons is due to the difficulty in combining two different glycosylated hemoglobin antigen epitope to produce a specific antibody. The present invention breaks away from the existing limitations in the related technology, uses hemoglobin common antigen as a first antigen, then uses hemoglobin and glycosylated hemoglobin specific antigen as molecular detecting layer for distinguishing hemoglobin and glycosylated hemoglobin, the sandwich immunoassay can not only achieve specificity, sensitivity, and stability repeatability, but also precisely detect the ratio between glycosylated hemoglobin and total hemoglobin on a single chip. This ratio is an important index of reference for clinically measuring average blood glucose concentration.

### SUMMARY OF THE INVENTION

An object of the present invention is to provide a chip for detecting glycosylated hemoglobin, so as to detect the content of glycosylated hemoglobin with respect to total hemoglobin in blood, thereby enhancing reliability and accessibility to blood glucose detection technology.

In order to achieve the abovementioned object, provided herewith is a chip for detecting glycosylated hemoglobin, comprising: a substrate; a biomolecular layer disposed on the substrate, and the biomolecular layer comprising a first anti-hemoglobin antibody.

In the structural design of the glycosylated hemoglobin detecting chip, the substrate herein comprises a plate and a modified layer, wherein, the substrate is preferably a rigid substrate or a flexible substrate. For which, the rigid substrate is preferably a glass substrate or silicon substrate; the flexible substrate is polydimethylsiloxane (PDMS), polystyrene, polypropylene, polymethylmethacrylate, polycarbonate, polyisobutylene, or any combination thereof, flexible substrate is preferably PDMS.

In the present invention, the modified layer disposed between the substrate and the bio-molecular layer can be made from a material selected from poly-lysine or other modifying materials, a primary function of the modifying layer is to keep away non-specific adhesion. In an aspect of the present invention, the modified layer used herein is made of fluoride having a polyelectrode layer, and its preparation procedure can be found in Anal. Chem. 82, 7804-7813, 2010. In a more detailed description, the modified layer in the present invention comprises, in an ascending order within structural context, a bipolar molecular layer, disposed on the substrate, the bipolar molecular layer has a hydrophilic distal end, and a hydrophobic distal end, the hydrophobic distal end is attached to the substrate; a cross-linking stack layer, disposed on top of the hydrophilic distal end of the bipolar molecular layer, and the cross-linking stack layer comprises at least a positively charged layer and at least a negatively charged layer; an attachment layer, disposed on the cross-linking stack layer; and a protein immobilization layer, disposed on the attachment layer.

In the glycosylated hemoglobin detecting chip of the present invention, the biomolecular layer includes anti-bodies, antigen ligand, receptor, or peptide, in addition to the first anti-hemoglobin antibody. The first anti-hemoglobin antibody binds the glycosylated hemoglobin and hemoglobin to the detecting chip before adding it to a biomolecular detecting layer for detecting hemoglobin and glycosylated hemoglobin concentration in a blood sample. Therefore, the biomolecular detecting layer comprises anti-glycosylated hemoglobin antibody, a second anti-hemoglobin antibody or other molecules capable of differentiating hemoglobin from glycosylated hemoglobin, then uses a non-specific second antibody of a light-emitting molecule to detect photo-intensity of the light-emitting molecule, so as to calculate a relative concentration of the glycosylated hemoglobin in the blood sample.

In addition, in order to achieve an object of the present invention, a method for detecting glycosylated hemoglobin is provided, comprising: (a) providing a chip for detecting glycosylated hemoglobin; and a bio-molecular layer, which is disposed on the substrate, and the bio-molecular layer comprises a first anti-hemoglobin antibody; (b) adding a blood specimen on the chip for detecting glycosylated hemoglobin to perform a combination of a hemoglobin and/or a glycosylated hemoglobin presented in the blood specimen and the chip for detecting glycosylated hemoglobin; (c) adding an anti-glycosylated hemoglobin antibody or a second anti-hemoglobin antibody to the chip for detecting glycosylated hemoglobin according to step (b), so as to perform a combination of the anti-glycosylated hemoglobin antibody and the glycosylated hemoglobin presented in the blood specimen, or a combination of the anti-hemoglobin antibody and the hemoglobin presented in the blood specimen; (d) adding a secondary antibody to the chip for detecting glycosylated hemoglobin according to step (c), so as to perform a combination of the secondary antibody and the anti-glycosylated hemoglobin, or a combination of the secondary antibody and the second anti-hemoglobin antibody, in which the secondary antibody is connected with a light emitting molecule; (e) providing a light source, which is irradiated on the chip for detecting glycosylated hemoglobin according to the step (d) to release an emitting light by an excitation of the light-emitting molecule; and (f) detecting intensity of the emitting light emitted by the light-emitting molecule according to step (e) through a detector.

In the method for detecting glycosylated hemoglobin of the present invention, step (b) can be performed through siphoning or dipping for delivering blood sample into the glycosylated hemoglobin detecting chip. Furthermore, another aspect of the present invention is to adopt step (b′) to filter the collected blood sample prior to adding blood sample to the detecting chip, for example: conducting a preliminary blood filtering process by a centrifugal means or chromatography for facilitating subsequent analysis after the sample and the chip are combined together.

In the method for detecting glycosylated hemoglobin of the present invention, step (c) is directed to adding separately anti-glycosylated hemoglobin antibody or second anti-hemoglobin to two different glycosylated hemoglobin detecting chip (the detecting chip is already loaded with blood sample), or, step (c) is directed to adding separately anti-glycosylated hemoglobin antibody or second anti-hemoglobin antibody to two different regions of the same detecting chip. (the detecting chip is already loaded with blood sample, to allow combination of the anti-glycosylated hemoglobin antibody and glycosylated hemoglobin in the blood sample, and, the second anti-hemoglobin antibody can be combined with the hemoglobin in the blood sample.

In the method for detecting glycosylated hemoglobin of the present invention, step (d) involves adding a secondary antibody with non-specificity with a light-emitting molecule attached thereto, calculating separately number of molecules based on the benefit of the excellent bonding between the secondary antibody with non-specificity and aforementioned anti-glycosylated hemoglobin or second anti-hemoglobin antibody, and the light intensity of the light-emitting molecule. Because the secondary antibody with non-specificity is attached to a light-emitting molecule, the light-emitting molecule will emit a radiating light when subject to light irradiation when a laser light shines upon the glycosylated hemoglobin. Therefore, it is preferable to add further a light-emitting testing reagent, such as chemical light-emitting testing reagent, so as to significantly increase light intensity of the light-emitting molecule. Therefore, the light-emitting molecule can be an enzyme which can form excellent bonding with secondary antibody with non-specificity, for example: horseradish peroxidase (HRP).

In the method for detecting glycosylated hemoglobin of the present invention, step (f) is done with using a detector to detect intensity of the emitting light according to step (e) from different regions of the same chip or from different chips. The detector can be, by way of example, a charge coupled detector (CCD) or photo detector. Content percentage of the glycosylated hemoglobin in blood can be found through calculation based on the emitting light intensity of the light emitting molecule measured from different regions or different detecting chips.

Therefore, the chip for detecting glycosylated hemoglobin of the present invention can combine hemoglobin and glycosylated hemoglobin with the detecting chip of the present invention, through the first anti-hemoglobin antibody of the biomolecular layer. Also the chip can identify glycosylated hemoglobin contained in hemoglobin, through the second anti-hemoglobin antibody and anti-glycosylated hemoglobin antibody, so as to obtain a content percentage of glycosylated hemoglobin in the blood. Based on the disclosure above, the present invention provides a chip for detecting glycosylated hemoglobin, using a biomolecular detecting layer and secondary antibody with non-specificity attached to a light-emitting molecule, so as to provide a reading on average blood glucose content in the last three months, and therefore obtain a more precise reading on average blood glucose concentration.

Other objects, advantages, and novel features of the invention will become more apparent from the following detailed description when taken in conjunction with the accompanying drawings.

### BRIEF DESCRIPTION OF THE DRAWINGS

FIG. 1 is a perspective diagram of a chip for detecting glycosylated hemoglobin according to the present invention.

FIG. 2 shows a placement of the glycosylated hemoglobin detecting chip in its detection setup.

FIG. 3 illustrates the way how a chip for detecting glycosylated hemoglobin of the present invention identifies the glycosylated hemoglobin and hemoglobin molecules.

### LIST OF NUMERAL REFERENCES

1 Chip for detecting glycosylated hemoglobin
11 Substrate
111 Substrate
112 Modified plate
12 Biomolecular layer
121 First anti-hemoglobin antibody
31 Pin
32 Chip for detecting
411 First detecting chip
412 Second detecting chip
432 Glycosylated hemoglobin
442 Anti-glycosylated hemoglobin antibody
452 Light-emitting molecule
421 First anti-hemoglobin antibody
431 Hemoglobin
441 Second anti-hemoglobin antibody
451 Secondary antibody with non-specificity

### DETAILED DESCRIPTION OF THE PREFERRED EMBODIMENT

The invention is capable of other embodiments and of being practiced or carried out in various ways. Variations and modifications of the foregoing are within the scope of the present invention. It will also be understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention.

### Preparative Example 1

### Preparing a Glycosylated Hemoglobin Detecting Chip Having a PDMS Flexible Substrate

The chip for detecting glycosylated hemoglobin in a blood sample according to the present invention, can enhance the preciseness and reliability in the practice detecting blood glucose, wherein the detecting chip is prepared by the following procedure:

First, modify PDMS with a fluoride having a polyelectrode layer (see for reference, Anal. Chem. 82, 7804-7813, 2010), which begins with coating 0.25% (w/v) PEI and 0.5% (w/v) PAA on an oxygen-plasma-activated substrate, and repeat for four times, so as to form a cross-linking stack layer. In the process of coating with PEI and PAA, clean the substrate with 20 ml of deionized water before coating the next layer. Wherein, additional 30 mg/ml of EDC and 10 mg/ml NHS (a cross-linking reagent) are added, to form an amide bond between the positively charged layer and the negatively charged layer. After the coating with PEI and PAA has been repeated four times, coat the uppermost layer with a PEI layer, to form a cross-linking stack layer having intertwiningly stacked positively charged layer (PEI) and negatively charged layer (PAA).

Subsequently, add pH 7.4, 1000 μg/ml of ACRL-PEG-NHS, to allow amide reaction between ACRL-PEG-NHS and PEI to form an attachment layer on the cross-linking stack layer for attaching a protein immobilization layer.

Next, add 15% (v/v) of FD dissolved in ethanol, 1% (v/v/) of AA dissolved in ethanol, and 1% (w/v) DPA photoinitiator, irradiate under room temperature with 365 nm of light for 40 minutes, then wash the surface of the chip with ethanol, and disposed in a nitrogen-filled environment for drying. Then, continue developing the chip surface for two hours using a mixed solution of 30 mg/ml EDC and 10 mg/ml NHS. After the washing is done, activated chip can be disposed in a G solution having 20 μg/ml protein for developing for 4 hours, to form an excellent bonding between protein G and AA.

Lastly, immersing the detecting chip having protein G in a 1 μg/ml goat anti-hemoglobin antibody (dissolved in a PBST buffer solution) for two hours, then wash the surface of the detecting chip with PBST buffer solution, so as to remove any first anti-hemoglobin antibody unattached on the detecting chip, in order to form a glycosylated hemoglobin detecting chip having first anti-hemoglobin antibody.

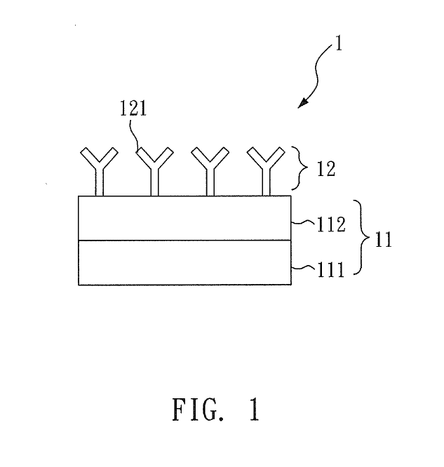

Accordingly, the present invention prepares a chip for detecting glycosylated hemoglobin prepared by the aforementioned method. As suggested in FIG. 1, the detecting chip 1 comprises a flexible substrate 111 disposed at the lowest bottom, a modified layer 112 disposed at the upper most portion of the flexible substrate 111; and a biomolecular layer 12 disposed on the substrate 11, wherein, the biomolecular layer 12 comprises a first anti-hemoglobin antibody 121, for use in combining glycosylated hemoglobin or hemoglobin molecule in a blood sample.

### Preparative Example 2

### Preparing a Glycosylated Hemoglobin Detecting Chip Having a Glass Substrate

The preparation procedure for this example is generally the same as for preparative example 1, with the exception being that the present preparation uses a glass substrate to replace the PDMS substrate in preparative example 1. Accordingly, the glycosylated hemoglobin detecting chip prepared by this example share the same structural arrangement as the glycosylated hemoglobin detecting chip prepared in preparative example 1, except that the constituting material of the substrate is different.

### Example 1

### Using Two Glycosylated Hemoglobin PDMS Detecting Chips

The glycosylated hemoglobin and hemoglobin in the blood sample can be combined to a detecting chip through the use of the first anti-hemoglobin antibody in the biomolecular layer of the glycosylated hemoglobin detecting chip prepared in the preparative example 1 of the present invention. Subsequently, add different antibodies (the first anti-hemoglobin antibody and anti-glycosylated hemoglobin antibody) separately into two detecting chips, so as to calculate separately on different detecting chips the number of hemoglobin and glycosylated hemoglobin in the blood sample. The concentration of the glycosylated hemoglobin in the blood sample can be calculated by using light-emitting and detecting devices to measure the number of glycosylated hemoglobin and hemoglobin combined on different chips. The detailed steps for carrying out the glycosylated hemoglobin detection method comprises the following:

First, use the glycosylated hemoglobin detecting chip as prepared in the preparative example 1 of the present invention for a detecting purpose Because the detecting chip comprises a first anti-hemoglobin antibody, glycosylated hemoglobin and hemoglobin in the blood sample can be sustained on the detecting chip. Therefore, the glycosylated hemoglobin detecting of the present invention can be used in detecting the concentration of glycosylated hemoglobin in the blood sample with respect to the total hemoglobin.

Next, collect blood samples from the test takers, and conduct preliminary filtering thereupon using filtering means well known in general practice, such as: centrifugal means or chromatography, so as to subject the blood sample for easier follow-on analysis.

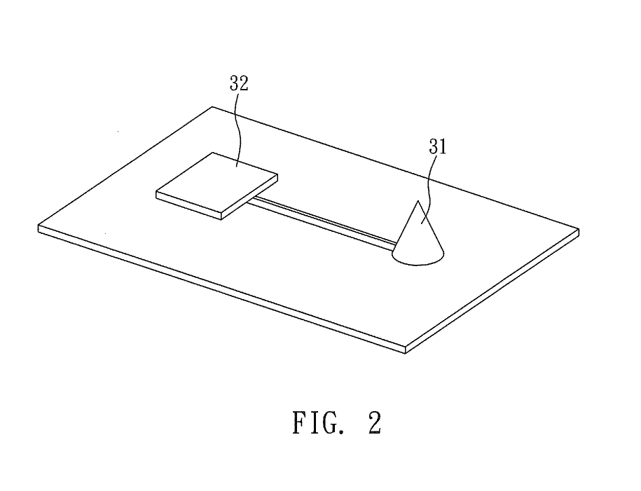

Then, draw the blood sample by siphoning onto a detecting chip. As shown in FIG. 2, the thin pin 31 is capable of penetrating through skin, causing 4 μl of blood sample to be drawn onto a detecting chip 32. In the meantime, the hemoglobin and glycosylated hemoglobin in the blood sample will form excellent bonding with goat anti-hemoglobin antibody (that is, first anti-hemoglobin antibody).

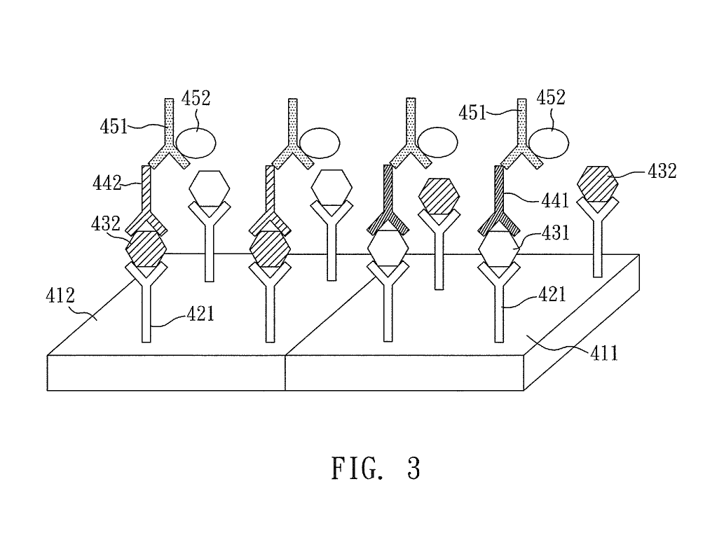

As shown in FIG. 3, after the hemoglobin 431 and the glycosylated hemoglobin 432 are combined onto the detecting chip 411, 412, through the first anti-hemoglobin 421, add 0.4 μg/ml mouse anti-hemoglobin antibody 441 (that is, second anti-hemoglobin antibody) dissolved in PBST buffer solution and mouse anti-glycosylated hemoglobin antibody 442 (that is, anti-glycosylated hemoglobin) onto different detecting chips 411, 412, so as to ensure that the anti-hemoglobin antibody 441 only form a specific bonding with hemoglobin 431 on the first detecting chip 411; furthermore, mouse anti-glycosylated hemoglobin antibody 442 can only form a specific bonding with glycosylated hemoglobin 432 on the second detecting chip 412.

Next, after the hemoglobin 431 and the glycosylated hemoglobin 432 separately form excellent bonding with the antibodies 441, 442, add 0.4 μg/ml mouse secondary antibody having non-specificity 451 dissolved in PBST buffer solution, the secondary antibody having non-specificity 451 is attached with a light-emitting molecule 452 of horseradish peroxidase (HRP) as a light-emitting enzyme.

On the account of the fact that, only mouse anti-hemoglobin antibody 441 is added to the chip 411 for the purpose of identifying hemoglobin 431 in the blood sample, the number of hemoglobin in the blood sample can be determined from the finding of the light intensity from the light-emitting molecule 452 on the detecting chip 411; only mouse anti-glycosylated hemoglobin antibodies 442 can be added to the chip 412, for the purpose of identifying glycosylated hemoglobin in the blood sample, the number of hemoglobin in the blood sample can be determined from the finding of the light intensity from the light-emitting molecule 452. Therefore, the mouse secondary antibody having non-specificity should be the antibody from the same host species as the second anti-hemoglobin antibody 441 and anti-glycosylated hemoglobin antibody 442, therefore the secondary antibody having non-specificity 451 only form excellent bonding with the aforementioned mouse antibodies 441, 442, and will not form bonding with the first anti-hemoglobin antibody 421 from different host species, securing the preciseness of the detection of the present invention.

Next, add chemical light-emitting enhancement reagent to the detecting chip, making the horseradish peroxidase to give off light chemically due to light irradiation. When two detecting chips are exposed to irradiating light of specific wavelength, the horseradish peroxidase (HRP) on the secondary antibody having non-specificity on the mouse will release an outgoing light due to light irradiation.

Then, use CCD (UVP, Bio-Imaging Systems, CA, USA) to capture an image, the number of glycosylated hemoglobin and hemoglobin can be determined through the light intensity obtained from the two detecting chips, and the content percentage of the glycosylated hemoglobin in the blood sample can be determined with Formula 1:

HbA1c(%)=HbA1c concentration/concentration of total hemoglobin×100% [Formula 1]

Wherein, HbA1c concentration can be replaced with in connection with the average light-emitting intensity of the light-emitting molecule on HbA1c, and the concentration of the total hemoglobin can be replaced with in connection with the average light-emitting intensity of the light-emitting molecule on hemoglobin.

### Experimental Group 1

### Using Two Glycosylated Hemoglobin

PDMS detecting chip for measuring the first sample Experimental group 1 is directed to measuring the concentration of hemoglobin in the blood sample through the abovementioned testing method, in the present experiment group, two glycosylated hemoglobin detecting chips (that is, the first detecting chip and the second detecting chip) are used for detection, but detection on the same chip but oriented to different antigenic determinant.

Wherein, after adding blood sample to the detecting chip, add the second anti-hemoglobin antibody and anti-glycosylated hemoglobin antibody separately to the first detecting chip and the second detecting chip. Afterwards, calculate the light intensity from the detecting chip based on the bonding between the non-specific secondary antibody attached with a light-emitting molecule and the second anti-hemoglobin antibody. Because the second anti-hemoglobin antibody can only identify the hemoglobin on the detecting chip, therefore, the total number of hemoglobin can be determined from calculating the light intensity from the detecting chip. Here, three repetitive experiments are performed, so as to individually obtain and calculate an average light intensity of HbA1c and hemoglobin.

### Experimental Group 2

### Using Two Glycosylated Hemoglobin PDMS Detecting Chips for Measuring the Second Ample

The experimental method contained in the present experimental group is identical to one adopted in experimental group 1, except that the present experimental group uses another blood sample. As shown by the experimental results from experimental group 1 and experimental group 2, and through Formula 1, a relative concentration (%) of the glycosylated hemoglobin used in experimental group 1 and 2 can be determined separately.

TABLE 1

| | | Average | |
|---|---|---|---|
| | Average HbA1c | Hemoglobin | HbA1c Relative |
| | Light-Emitting | Light-Emitting | Concentration |
| | Intensity | Intensity | (%) |
| Experimental | 1365476 | 5976266 | 23 |
| Group 1 | | | |
| Experimental | 3023474 | 5124533 | 59 |
| Group 2 | | | |

### Example 2

### Using a Single Glycosylated Hemoglobin PDMS Detecting Chip

The present example uses the glycosylated hemoglobin PDMS detecting chip prepared by the preparative example 1 of the present invention. The method of measurement is identical to the one demonstrated in example 1, with the exception that example 1 uses two detecting chips, for separately detecting glycosylated hemoglobin and hemoglobin content; the present example uses only one chip, conducting detection on the same chip but different antigenic determinant. This means that one single chip is divided into two regions, for the purpose of detecting amount of glycosylated hemoglobin and hemoglobin individually.

### Example 3

### Using Two Glycosylated Hemoglobin Glass Detecting Chip

### Experimental Group 3

### Using Two Glycosylated Hemoglobin Glass Detecting Chip for Measuring the First Sample

The present example is directed to using the glycosylated hemoglobin glass detecting chip prepared in the preparative example 2 of the present invention; the method of detection is the same as shown in example 1.

After blood samples are added to two detecting chips, add individually the second anti-hemoglobin antibody and anti-glycosylated hemoglobin antibody separately to two detecting chips, and add a secondary antibody with non-specificity having a light-emitting molecule attached thereto, so as to determine the average light intensity of HbA1c and hemoglobin in the blood sample based on the calculation of the light intensity of the detecting chip. The ensuing results are shown below.

TABLE 2

| | | Average | |
|---|---|---|---|
| | Average HbA1c | Hemoglobin | HbA1c Relative |
| | Light-Emitting | Light-Emitting | Concentration |
| | Intensity | Intensity | (%) |
| Experimental | 1294768 | 5629429 | 23 |
| Group 3 | | | |

To summarize the above disclosure, the chip for detecting glycosylated hemoglobin of the present invention provides a solution made possible by the combination between a biomolecular layer having antibody with specificity and glycosylated hemoglobin and hemoglobin I in the blood sample, followed by addition of antibodies capable of identifying glycosylated hemoglobin and hemoglobin and the addition of a secondary antibody having a light-emitting molecule. To a more elaborate degree, a relative concentration of the glycosylated hemoglobin in the blood sample can be determined based on the number of anti-glycosylated hemoglobin antibody and second anti-hemoglobin antibody in the blood sample. Therefore, the present invention provides a detecting chip and a detecting method suitable for detecting HbA1cx in a blood sample, thereby increasing accessibility to household healthcare for diabetic patients.

Although the present invention has been explained in relation to its preferred embodiment, it is to be understood that many other possible modifications and variations can be made without departing from the spirit and scope of the invention as hereinafter claimed.

What is claimed is:

1. A chip for detecting glycosylated hemoglobin, comprising:
a substrate; and
a biomolecular layer, which is disposed on the substrate, and the bio-molecular layer comprises a first anti-hemoglobin antibody.

2. The chip for detecting glycosylated hemoglobin as claimed in claim 1, wherein the substrate comprises a plate and a modified layer, and the modified layer is disposed between the substrate and the bio-molecular layer.

3. The chip for detecting glycosylated hemoglobin as claimed in claim 2, wherein the substrate is a rigid substrate or a flexible substrate.

4. The chip for detecting glycosylated hemoglobin as claimed in claim 3, wherein the substrate is a glass substrate.

5. The chip for detecting glycosylated hemoglobin as claimed in claim 3, wherein the flexible substrate is a PDMS substrate.

6. A method for detecting glycosylated hemoglobin, comprising the following steps:
(a) providing a chip for detecting glycosylated hemoglobin, wherein the chip for detecting glycosylated hemoglobin comprises: a substrate; and a bio-molecular layer, which is disposed on the substrate, and the bio-molecular layer comprises a first anti-hemoglobin antibody;
(b) adding a blood specimen on the chip for detecting glycosylated hemoglobin to perform a combination of a hemoglobin and/or a glycosylated hemoglobin presented in the blood specimen and the chip for detecting glycosylated hemoglobin;
(c) adding an anti-glycosylated hemoglobin antibody or a second anti-hemoglobin antibody to the chip for detecting glycosylated hemoglobin according to step (b), so as to perform a combination of the anti-glycosylated hemoglobin antibody and the glycosylated hemoglobin presented in the blood specimen, or a combination of the anti-hemoglobin antibody and the hemoglobin presented in the blood specimen;
(d) adding a secondary antibody to the chip for detecting glycosylated hemoglobin according to step (c), so as to perform a combination of the secondary antibody and the anti-glycosylated hemoglobin, or a combination of the secondary antibody and the second anti-hemoglobin antibody, in which the secondary antibody is connected with a light emitting molecular;
(e) providing a light source, which is irradiated on the chip for detecting glycosylated hemoglobin according to step (d) to release a emitting light by an excitation of the light-emitting molecular; and
(f) detecting a strength of the emitting light emitted by the light-emitting molecular according to step (e) through a detector.

7. The method for detecting glycosylated hemoglobin as claimed in claim 6, which further comprises a step (g): calculating the strength of the emitting light obtained from adding the anti-glycosylated hemoglobin antibody and anti-hemoglobin antibody respectively according to step (c), so as to calculate a content percentage of the glycosylated hemoglobin relative to the hemoglobin, in which the content percentage calculated by the following formula (2) is obtained by comparing a standard method,
%HPLC(standard)=A*%the method of the present invention+B, formula (2)
wherein A=0.1, B=3.3.

8. The method for detecting glycosylated hemoglobin as claimed in claim 6, wherein the light emitting molecular is an enzyme.

9. The method for detecting glycosylated hemoglobin as claimed in claim 8, wherein the enzyme is horseradish peroxidase (HRP).

10. The method for detecting glycosylated hemoglobin as claimed in claim 6, where in the first anti-hemoglobin antibody and the second anti-hemoglobin antibody are different species antibodies respectively.

11. The method for detecting glycosylated hemoglobin as claimed in claim 6, wherein the first anti-hemoglobin antibody and the anti-glycosylated hemoglobin antibody are different species antibodies respectively.

12. The method for detecting glycosylated hemoglobin as claimed in claim 6, wherein the detector is a charge-coupled device (CCD) and an optical detection instrument.

13. The method for detecting glycosylated hemoglobin as claimed in claim 6, wherein the substrate comprises a plate and a modified layer, and the modified layer is disposed between the substrate and the bio-molecular layer.

14. The method for detecting glycosylated hemoglobin as claimed in claim 13, wherein the substrate is a rigid substrate or a flexible substrate.

15. The method for detecting glycosylated hemoglobin as claimed in claim 14, wherein the substrate is a glass substrate.

16. The method for detecting glycosylated hemoglobin as claimed in claim 14, wherein the flexible substrate is a PDMS substrate.

17. The method for detecting glycosylated hemoglobin as claimed in claim 6, which further comprises a step (b′) before the step (b),
(b′) filtrating the blood specimen.

18. The method for detecting glycosylated hemoglobin as claimed in claim 6, in the step (b), the blood specimen is added into the chip for detecting glycosylated hemoglobin by the method of siphoning or moistening.

19. The method for detecting glycosylated hemoglobin as claimed in claim 6, which further comprises a step (d′) after the step (d),
(d′) adding a light inducing agent.
